SEQUENCE LISTING (3 sequences)

SEQ ID NO: 1 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 2 - DNA, 3383 bp, Homo sapiens
gctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgctgcggccgcctgggccctttcctccctcaagcccatctccacaacctcgagcctgggctctggtccactactgccccagaccaccctcaaagctggtcttcagaaatcaataatatgagtttttattttgtttttttttttttttttgtagtttattttggagtctagtatttcaataatttaagaatcagaagcactgacctttctacattttataacattattttgtatataat

SEQ ID NO: 3 - protein, 684 aa, Artificial Sequence
  extra amino acids in the XB construct
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKWRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

(19) United States
(12) Patent Application Publication
(10) Pub. No.: US 2007/0213266 A1
(43) Pub. Date: Sep. 13, 2007

(54) Methods for using and identifying modulators of Delta-like 4

(75) Inventors: Parkash Gill, Agoura Hills, CA (US); Antonio Duarte, Lisboa (PT); Weidong Jiang, Cupertino, CA (US)

Correspondence Address: FISH & NEAVE IP GROUP;ROPES & GRAY LLP, ONE INTERNATIONAL PLACE, BOSTON, MA, 02110-2624, US

(73) Assignee: VasGene Therapeutics, Inc., Broomall, PA (US)

(21) Appl. No.: 11/514,773

(22) Filed: Sep. 1, 2006

Related U.S. Application Data
(60) Provisional application No. 60/713,637, filed on Sep. 1, 2005.

(51) Int. Cl. A61K 38/18; G01N 33/574

(52) U.S. Cl. 514/12; 435/7.23

(57) ABSTRACT

In certain embodiments, this present invention provides methods of identifying and using agonists and antagonists of Delta-like 4 (Dll4) signaling.

### CROSS-REFERENCE TO RELATED APPLICATIONS

This application claims the benefit of U.S. Provisional Application Ser. No. 60/713,637, filed Sep. 1, 2005. All the teachings of the above-referenced application is incorporated herein by reference.

### BACKGROUND OF THE INVENTION

Angiogenesis, the development of new blood vessels from the endothelium of a preexisting vasculature, is a critical process in the growth, progression, and metastasis of solid tumors within the host. During physiologically normal angiogenesis, the autocrine, paracrine, and amphicrine interactions of the vascular endothelium with its surrounding stromal components are tightly regulated both spatially and temporally. Additionally, the levels and activities of proangiogenic and angiostatic cytokines and growth factors are maintained in balance. In contrast, the pathological angiogenesis necessary for active tumor growth is sustained and persistent, representing a dysregulation of the normal angiogenic system. Solid and hematopoietic tumor types are particularly associated with a high level of abnormal angiogenesis. More recently, it has become apparent that certain types of leukemia are also influenced by signaling involved in angiogenesis.

Agents that inhibit angiogenesis are useful in treating cancer. Avastin™ (bevacizumab), a monoclonal antibody that binds to Vascular Endothelial Growth Factor (VEGF), has proven to be effective in the treatment of a variety of cancers. Antagonists of the SDF/CXCR4 signaling pathway inhibit tumor neovascularization and are effective against cancer in mouse models (Guleng et al. Cancer Res. 2005 Jul. 1; 65(13):5864-71). The isocoumarin 2-(8-hydroxy-6-methoxy-1-oxo-1H-2-benzopyran-3-yl) propionic acid (NM-3) has completed phase I clinical evaluation as an orally bioavailable angiogenesis inhibitor. NM-3 directly kills both endothelial and tumor cells in vitro and is effective in the treatment of diverse human tumor xenografts in mice (Agata et al. Cancer Chemother Pharmacol. 2005 Jun. 10; [Epub ahead of print]).

Angiogenesis is a feature of other, non-neoplastic disorders. Various ocular disorders, particularly proliferative retinopathies and age-related macular degeneration, and inflammatory disorders, such as rheumatoid arthritis and psoriasis, are marked by increased vascularization of the affected tissue. Anit-angiogenic agents are effective for the treatment of these disorders. Macugen™, an aptamer that binds to VEGF has proven to be effective in the treatment of neovascular (wet) age-related macular degeneration. The success of TNF-alpha antagonists in the treatment of rheumatoid arthritis is partially attributed to anti-angiogenic effects on the inflamed joint tissue (Feldmann et al. Annu Rev Immunol. 2001; 19:163-96).

Arteriogenesis, a process related to but distinct from angiogenesis, occurs when the lumen of a pre-existing vessel increases to form a collateral. After myocardial infarction or peripheral ischemia (e.g., limb, kidney, etc.) arterioles become more significant conductance vessels in order to maintain blood flow after occlusion of the major artery serving the affected tissue. Thus, agents that promote arteriogenesis may be used to treat myocardial infarction and other ischemic events, and may also be used to prevent an ischemic event where a partial arterial occlusion is detected or suspected.

The Notch pathway, and particularly Notch1 and Notch4, participates in angiogenic processes. Notch signalling is generally involved in the regulation of processes as diverse as cellular proliferation, differentiation, specification and survival (Artavanis-Tsakonas et al., 1999). Its complexity in vertebrates is illustrated by the existence of multiple Notch receptor and ligands, each with distinct patterns of expression. In mammals there are four Notch receptors (notch1-4) and five ligands (jagged1, 2 and Dll1, 3 and 4). Mutations of Notch receptors and ligands in mice lead to abnormalities in various organs, from all three germ lines, including the vascular system (Iso et al., 2003). The Notch pathway functions through local cell interactions, the extracellular domain of the ligand, present on the surface of one cell, interacts with the extracellular domain of the receptor on an adjacent cell. This interaction allows the action of two ADAM proteases on the extracellular domain of Notch followed by the action of a γ-secretase on the transmembrane domain releasing the intracellular domain from the cell membrane and allowing it to be directed to the nucleus, where it functions with CSL to activate the expression of transcriptional repressors of the enhancer-of-split family (Mumm & Kopan, 2000).

Arterial versus venous differentiation has long been thought to be mainly dependent on physical factors such as blood pressure and oxygen concentration. Recently, however, the identification of a number of genes that are specifically expressed in arterial or venous endothelial cells well before the onset of circulation, seems to indicate an important role for genetic determination of endothelial cells in the primary differentiation events between arteries and veins. Among these genes are eph-B4, specifically expressed in venous endothelial cells (Adams et al., 1999) and ephrin-B2 (Adams et al., 1999; Gale et al., 2001), notch1 (Krebs et al., 2000), notch4 (Uyttendaele et al., 1996) and dll4 (Shutter et al., 2000), among others, which are specifically expressed in arterial endothelial cells.

Studies with mutations in zebrafish Notch homologues demonstrate the importance of this pathway in regulating the arterial versus venous endothelial differentiation, downstream of vascular endothelial growthfactor and sonic-hedgehog and upstream of the ephrin pathway (Lawson et al., 2002), being the earliest genes expressed in an endothelial arterial specific fashion. There is mounting evidence, in both zebrafish and mouse, that Notch function is essential in the establishment of the arterial endothelial cell fate (Lawson et al., 2002; Fischer et al., 2004; Duarte et al., 2004).

It is a goal of the present disclosure to provide agents and therapeutic treatments for modulating angiogenesis, arteriogenesis and vessel identity.

### SUMMARY OF THE INVENTION

In certain aspects, the disclosure provides uses for, and methods for identifying, agonists and antagonists of the Notch ligand Delta-like 4 (Dll4). Surprisingly, as taught herein, both agonists and antagonists of Dll4 may be used to treat tumors undergoing angiogenesis or in other situations where it is desirable to inhibit or disrupt angiogenesis. Furthermore, the disclosure provides methods for stimulating arteriogenesis by administering a Dll4 agonist. Arteriogenesis is the process of collateral artery formation and growth, typically in ischemic tissues. Thus Dll4 agonists may be used to treat patients suffering from, or at risk for, an ischemic event, such as a peripheral or coronary ischemia. The disclosure further relates to the discovery that upregulation of Dll4 causes endothelial cells to adopt an arterial identity, while inhibition of Dll4 causes endothelial cells to adopt a venous identity. Thus, the disclosure provides methods for altering venous or arterial identity by using, as appropriate an agonist or antagonist of Dll4. Additionally, the disclosure provides biomarkers that may be used to assess whether an agent of interest is an agonist or antagonist of Dll4 signaling.

The disclosure further demonstrates that a monomeric polypeptide comprising a portion of the extracellular domain of Dll4 promotes angiogenesis at low concentrations and inhibits VEGF-mediated angiogenesis at higher concentrations. Soluble Dll4 polypeptide promotes arterialization or arteriogenesis at all concentrations. Accordingly, by selecting the appropriate dose of monomeric soluble Dll4 polypeptide, differing effects on angiogenesis may be achieved. In certain embodiments, a soluble Dll4 polypeptide comprises the DSL domain of SEQ ID NO:1 (amino acids 173-233) but lacks the transmembrane and intracellular portions (amino acids 552-685). Optionally, the Dll4 polypeptide comprises at least 200 amino acids in the region of amino acids 27-528 of SEQ ID NO:1. Optionally, the Dll4 polypeptide comprises amino acids 27-486 of SEQ ID NO:1 and preferably amino acids 27-524. In certain embodiments, the soluble Dll4 polypeptide includes a moiety that confers desirable pharmacokinetic properties, such as an Fc domain or a polyoxyalkylene moiety (e.g., PEG).

In certain embodiments, the disclosure provides methods for stimulating arteriogenesis. Such methods may comprise administering to a subject in need thereof, an effective amount of an agonist of Dll4 signaling. The subject may have or be at risk for an ischemic condition. The subject may have coronary artery disease, including, for example, angina or may have had a myocardial infarction. The subject may have a peripheral artery disease, such as an ischemic event or partial occlusion in a limb, the brain or an organ, such as the kidney. The subject may be diagnosed as being at risk for an ischemic event.

In certain embodiments, the disclosure provides methods for promoting the adoption of arterial characteristics in a blood vessel. Such a method may comprise administering to a blood vessel ex vivo or to a subject in need thereof, an effective amount of an agonist of Dll4 signaling. The blood vessel may be a venous graft, such as a saphenous vein graft.

In certain embodiments, the disclosure provides methods for inhibiting angiogenesis, the method comprising, administering to a subject in need thereof, an effective amount of an antagonist of Dll4 signaling. The subject may have an angiogenesis-associated disease. Examples of angiogenesis-associated diseases include angiogenesis-dependent cancer, benign tumors, inflammatory disorders, chronic articular rheumatism and psoriasis, ocular angiogenic diseases, Osler-Webber Syndrome, myocardial angiogenesis, plaque neovascularization, telangiectasia, hemophiliac joints, angiofibroma, wound granulation, wound healing, telangiectasia psoriasis scleroderma, pyogenic granuloma, rubeosis, arthritis and diabetic neovascularization. A method may further include administering at least one additional anti-angiogenesis agent that inhibits angiogenesis. Such additional agent may be used in an additive or synergistic manner with the antagonist of Dll4 signaling.

In certain embodiments, the disclosure provides methods for disrupting angiogenesis. Such methods may comprise administering to a subject in need thereof, an effective amount of an agonist of Dll4 signaling.

In certain embodiments, the disclosure provides methods for disrupting tumor vasculature. Such methods may comprise administering to a subject in need thereof, an effective amount of an agonist of Dll4 signaling.

In certain embodiments, the disclosure provides methods for evaluating the effects of a test agent on Dll4 signaling. A method may comprise (a) contacting a cell of endothelial lineage with the test agent; and (b) detecting a phenotype associated with arterial or venous phenotype. A test agent that promotes the adoption of an arterial phenotype or an agent that inhibits the adoption of a venous phenotype is an agonist of Dll4 signaling, while a test agent that inhibits the adoption of an arterial phenotype or promotes the adoption of a venous phenotype is an antagonist of Dll4 signaling.

The disclosure provides characteristics that may be used to distinguish agonists and antagonists of Dll4 signaling. In general, agonists of Dll4 signaling stimulate, in a mammalian endothelial cell, expression of an arterial phenotype and inhibit expression of a venous phenotype. In general, antagonists of Dll4 signaling inhibit, in a mammalian endothelial cell, expression of an arterial phenotype and stimulate expression of a venous phenotype. Any known feature that distinguishes arterial and venous endothelial cells may be detected for the purpose of assessing arterial and venous phenotypes. For example, expression of EphrinB2 and expression of connexin37 may be used as indicators of arterial phenotype. As another example, expression of EphB4 may be used as an indicator of venous phenotype.

### BRIEF DESCRIPTION OF THE DRAWINGS

FIG. 1 shows the amino acid sequence of the human Delta-like 4 protein (SEQ ID NO:1; GenBank NP_{—}061947). The signal sequence, amino acids 1-26, is underlined. The transmembrane domain, amino acids 532-552, is bolded. The extracellular domain of the mature protein is amino acids 27-531, although imprecision in signal peptide processing may result in a protein that is slightly longer or shorter. The intracellular domain is amino acids 532-685.

FIG. 2 shows the nucleic acid sequence (cDNA) encoding the human Delta-like 4 protein (SEQ ID NO:2; GenBank NM_{—}019074). The coding sequence is nucleic acids 321-2378.

FIG. 3. pZ/EG-mDll4 transgenesis vector and result of Cre activity.

FIG. 4. (a) LacZ staining of a ZEG-mDll4 embryo at E8.0; (b) EGFP expression in the dt embryos at E8.5. (c) haemorrhaging and pericardial edema in dt embryos at E9.0.

FIG. 5. Wholemount PECAM1 immunostaining of E9.0 and E9.5 dt and control embryos. (a) control embryo at E9.0, (b) dt embryo at E9.0 showing a hypertrophied dorsal aorta (lower left arrow), ramified ACV (lower right arrow) and an immature vascular plexus in the head region (upper arrow) (c) control embryo at E9.5, (d) dt embryo at E9.5 showing hypertrophied dorsal aorta and almost no sign of an ACV, immature vascular plexus in the head region and hypertrophied sinus venosus and heart ventricle. Half sectioning the stained embryos at E9.5 showed that the aorta of the dt embryos (f) atrophies just posterior to its connection to the sinus venosus (lower arrow), while in the control embryo (e) remains with the same calibre throughout the embryo. The intersomitic vessels (upper arrow) of the dt embryos (h) appear slightly dilated and shorter than those of control embryos (g). In the dorsal region (lower arrow) of the dt embryos angiogenesis fails to occur. (i) yolk sac of a E9.5 control embryo, (j) yolk sac of a dt embryo showing lack of remodelling of the primary plexus in contrast to the highly organized structure of the vasculature in the control embryos.

FIG. 6. PECAM1 immunostaining in cryosections and microangiography. (a-g) serial sections of a E9.5 dt embryo (anterior-posterior) showing fusion between the aorta (upper right arrow) and the ACV (upper left arrow) just prior to its connection to the sinus venosus (lower arrow). In section (a) the ACV consists of a plexus of small capillaries (upper left arrow) which join to form a single vessel with a large lumen just prior to its fusion with the dorsal aorta. Section (e) shows the aortic atrophy in regions posterior to the sinus venosus. (f,g) serial sections of a E9.5 wild type embryo depicting the same regions stated above. Microangiography with India ink injection confirmed the existence of functional connections between the dorsal aortae and the ACV of dt embryos (i), with ink flowing directly from the aortae (left hand arrow) to the sinus venosus (right hand arrow), in contrast to the regular flow observed in the control embryos (h).

FIG. 7. Venous expression of arterial markers in dt embryos. In situ hybridization of cryosections from E9.0 dt embryos.with ephrin-B2(a,b,c) and connexin-37 (d,e,f) specific riboprobes. The mutant embryos show concomitant expression of these arterial specific markers in the both the dorsal aortae (AD) and anterior cardinal veins (VCA) In the control embryos (c,f), as expected, the expression is restricted to the aortae.

FIG. 8. Upregulation of Notch signalling in the venous endothelium of the mutant embryos. In situ hybridization of cryosections from E9.0 dt embryos.with hey1 (a,b,c) and Notch1 (d,e,f) specific riboprobes. Both genes appear upregulated in the anterior cardinal veins (VCA). In the control embryos (c,f), as expected, the expression is restricted to the aortae.

FIG. 9. Downregulation of venous specific markers in dt embryos. In situ hybridization and immunostainings of cryosections from E9.0 dt embryos, (a) anti-Eph-B4 immunostain, (c) eph-b4 mRNA, and E9.0 control embryos, (b) anti-Eph-B4 immunostain, (d) eph-b4 mRNA.

FIG. 10. Shows a schematic of the human Dll4 domain structure (top) and an annotated human Dll4 amino acid sequence (SEQ ID NO:1) (bottom). The signal sequence and DSL domain are underlined and indicated. The eighth EGF8 domain (EGF8) is shaded. The ΔXB (ΔEGF8) construct contains 19 extra amino acids (RSPSCIYRRSWRSRGAQIL) (SEQ ID NO:3) at the C-terminus after the CAS residues of the EGF8 repeat. The P524-His construct ends at P524, 4 amino acids before the transmembrane domain, with a 6×His tag at the C-terminus. Both constructs contain the receptor-binding domain, DSL domain. Full length constructs have either a Myc tag or no tag.

FIG. 11. Shows the purified hDll4-P524-6×His protein (histidine tagged hDll4-P524) after nickel column purification (SDS-PAGE: CBB-G250 Staining).

FIG. 12. hDll4 inhibits tube formation in human arterial endothelial cells (HUAEC). VEGF was used at 20 ng/ml as a positive control. Dll4 at lower concentrations (50 ng/ml or 100 ng/ml) promoted tube formation, while Dll4 at 500 ng/ml inhibited tube formation.

FIG. 13. hDll4 inhibits sprouting in human arterial endothelial cells (HUAEC). VEGF was used at 20 ng/ml as a positive control. Dll4 at 100 ng/ml or 200 ng/ml promoted sprouting, while Dll4 at 500 ng/ml inhibited sprouting.

FIG. 14. hDll4 inhibits VEGF-stimulated sprouting in human arterial endothelial cells (HUAEC) at high concentrations. VEGF was used at 20 ng/ml. Dll4 at 100 ng/ml had little effect, while Dll4 at 200 ng/ml inhibited VEGF-stimulated sprouting.

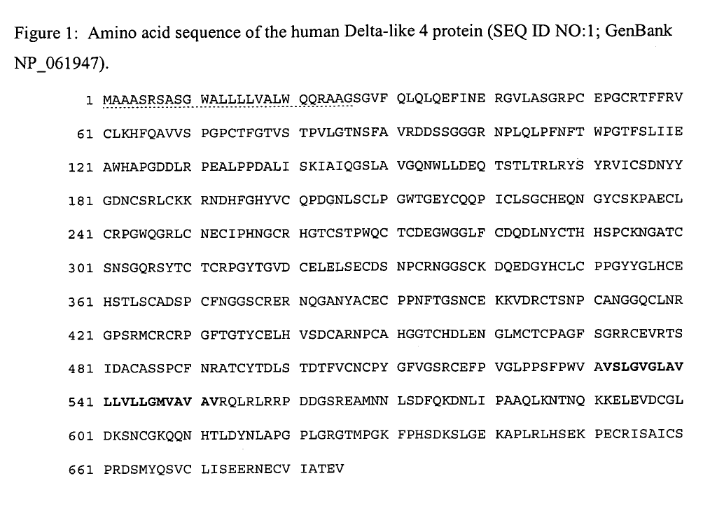

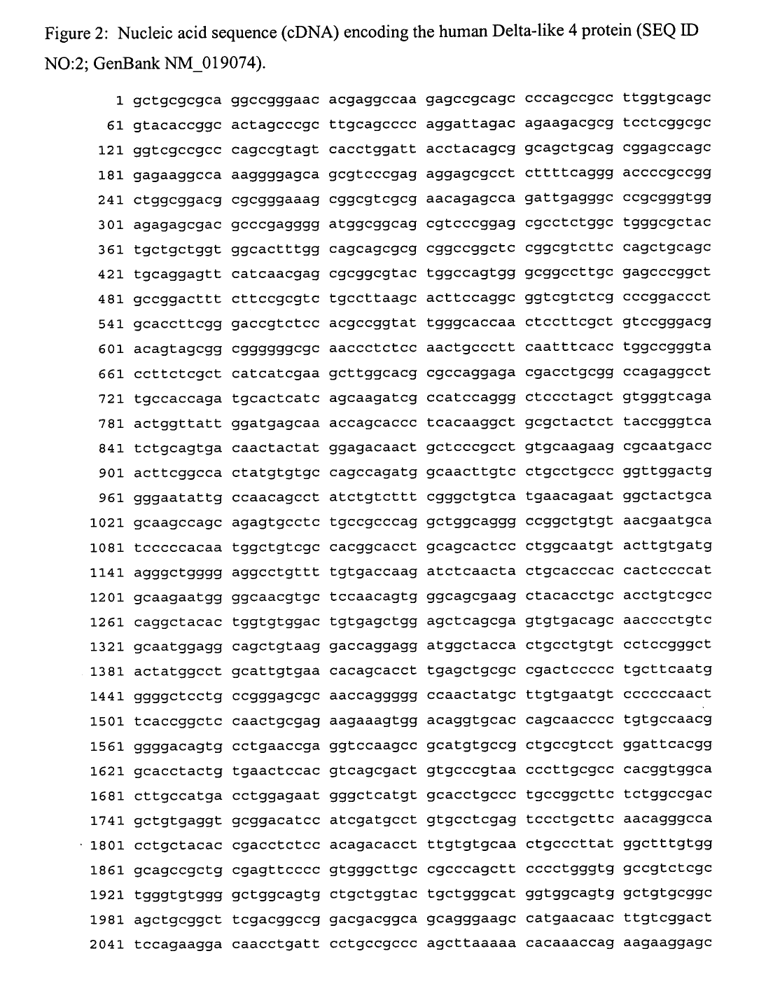

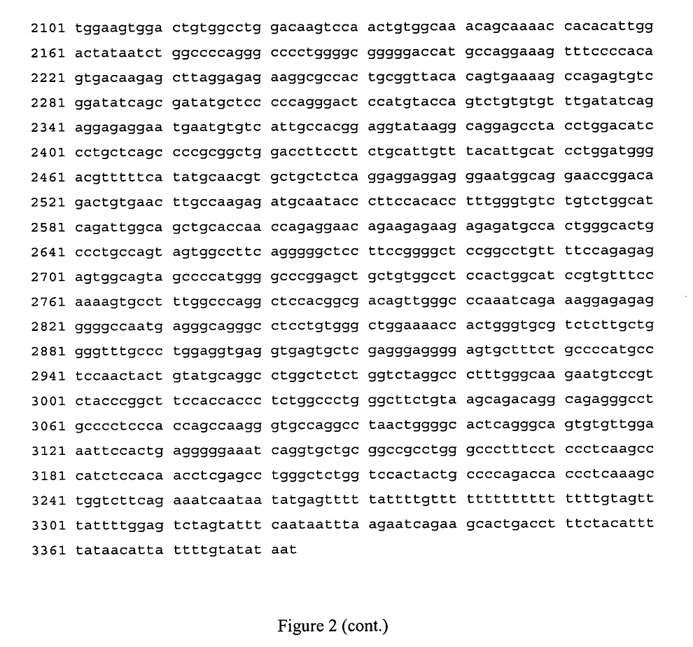

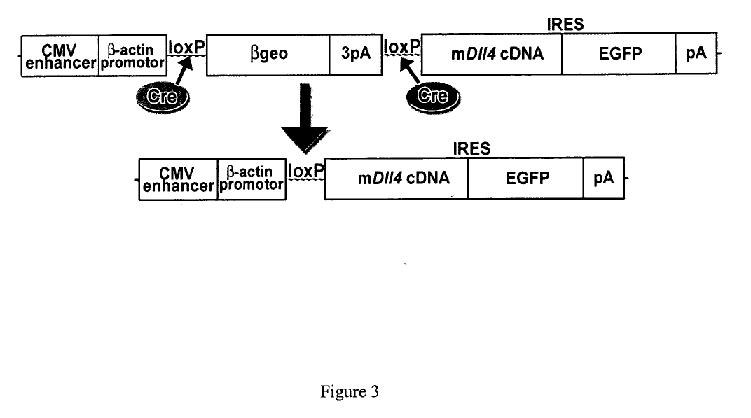

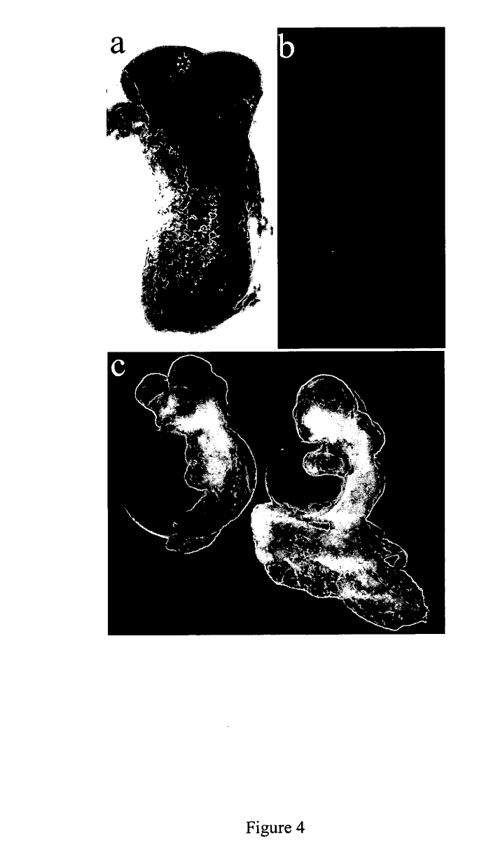

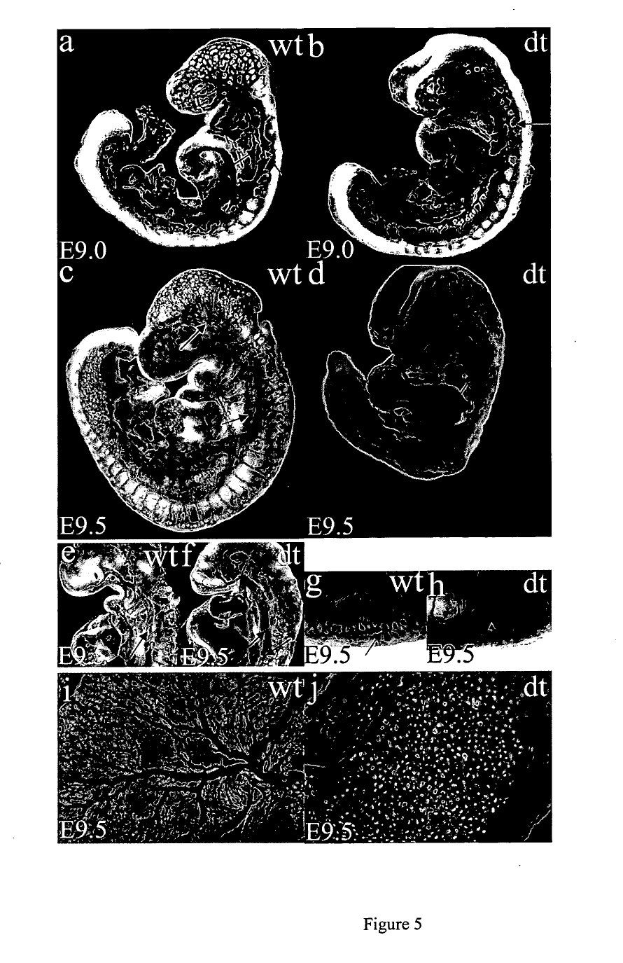

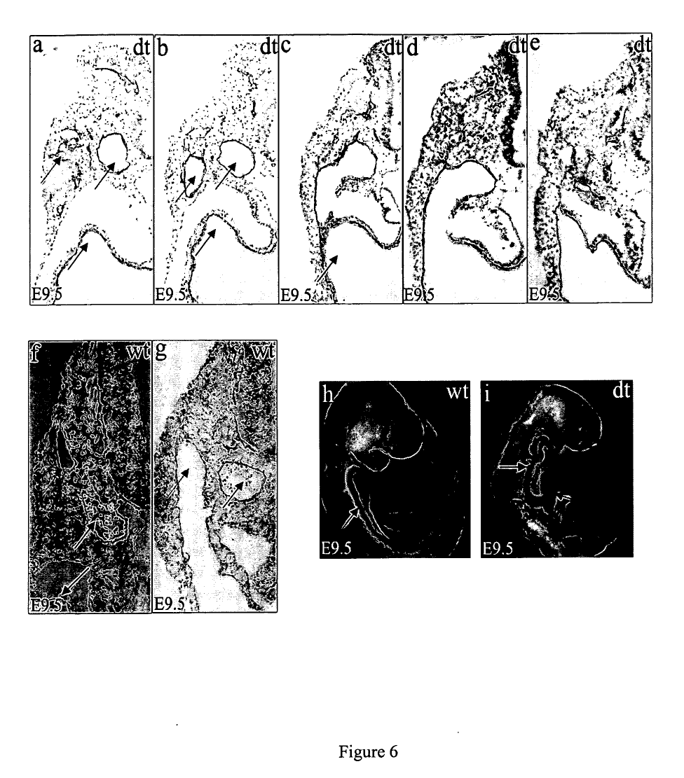

### DETAILED DESCRIPTION OF THE INVENTION

The current invention is based in part on the discovery that Delta-like 4 function is essential for angiogenesis in vivo, and, moreover, that an increase of Delta-like 4 activity is associated with increased proliferation of arterial endothelial cells and an increased adoption of an arterial identity by endothelial cells. Applicants generated mouse Dll4 knockout mutations that evinced dosage sensitive defects in angiogenesis. Furthermore, Applicants generated Dll4 overexpression models in mouse and demonstrated that increased expression of Dll4 causes, in some instances, hypertrophy of arterial tissue and, moreover, causes venous tissue to adopt an arterial identity. Based on these results, it is apparent that angiogenesis, in which a system of arterial and venous microvessels is generated, is highly sensitive to Dll4 activity and may be perturbed (e.g., inhibited or caused to occur in a disorganized or ineffective manner) by inhibition or hyperactivation of Dll4. Thus, surprisingly, both agonists and antagonists of Dll4 may be used to treat tumors undergoing angiogenesis. Furthermore, the invention relates to the discovery that overexpression of Dll4 can stimulate arterial growth, and may therefore be used to stimulate arteriogenesis. Arteriogenesis is the process of collateral artery formation and growth, typically in ischemic tissues. Thus Dll4 agonists may be used to treat patients suffering from, or at risk for, an ischemic event, such as a peripheral or coronary ischemia. Furthermore, the dislcosure demonstrates that a soluble monomeric Dll4 polypeptide can act to inhibit or promote angiogenesis at low or high concentrations, respectively.

The invention further relates to biomarkers that may be used to assess whether an agent of interest is an agonist or antagonist of Dll4 signaling. The scientific literature relating to Delta proteins generally, including Dll4, provides no clarity as to whether a particular agent activates or inhibits Dll4-mediated signaling. For example, Dll4 and Delta extracelluar domains (e.g., soluble monomeric forms, forms with deleted intracellular domains, and soluble Fc fusions) have been tested in a variety of assays and it remains unclear whether any of the observed effects are due to agonist or antagonist activity, or whether there is any meaningful activity at all. Moreover, reagents may affect Dll4 signaling in a variety of ways. For example, a reagent may affect Notch 1 and/or Notch 4 activation, or activation of retrograde Dll4 signaling, possibly mediated by the Dll4 intracellular domain. A reagent may also affect the activity of preseniline protease activity, which may affect both Notch1 and Notch4. The present disclosure demonstrates that Dll4 hyperactivation causes endothelial cells to adopt an arterial phenotype, typified by expression of EphrinB2 and connexin37, while Dll4 loss of function causes endothelial cells to adopt a venous identity, typified by expression of EphB4. This information about the genetically-determined, in vivo effects of Dll4 activity will permit the identification of both known and newly discovered agents as agonists or antagonists of Dll4 signaling.

Accordingly, in certain aspects, the disclosure provides numerous polypeptide compounds (agents) that may be used to treat cancer as well as angiogenesis related disorders and unwanted angiogenesis related processes.

Dll4 is a Notch ligand and contains a signal sequence, a DSL domain, eight epidermal growth factor-like repeats, a transmembrane domain, and an intracellular region, all of which are characteristics of members of the Delta protein family. The tissue distribution of Delta-4 mRNA resembles that previously described for Notch-4 (Int-3) transcripts. Soluble forms of the extracellular portion of Delta-4 inhibit the apparent proliferation of human aortic endothelial cells, but not human pulmonary arterial endothelial cells. Yoneya et al. J. Biochem. Vol. 129, pp. 27-34 (2001).

Members of the Notch family of proteins are transmembrane receptors that contain characteristic multiple epidermal growth factor (EGF)-like repeats as well as conserved domains such as RAM, ankyrin-like repeat, and PEST sequences. Ligands for Notch proteins include Delta and Serrate in Drosophila melanogaster, LAG-2 and APX-1 in Caenorhabditis elegans, and Delta and Serrate (or Jagged) in vertebrates. These ligands are also transmembrane proteins and contain a highly conserved DSL (Delta-Serrate-LAG-2) motif upstream of a variable number of EGF-like repeats. The DSL domain is a characteristic feature of Notch ligands and is important for protein function; thus, point mutation of the DSL domain in LAG-2 results in a loss of activity. Although the Delta and Jagged (Serrate) proteins of vertebrates exhibit similar structures, each group of proteins also possesses several distinct features. Thus, whereas vertebrate Delta proteins contain eight EGF-like repeats, Jagged proteins contain 16 such repeats. Furthermore, the EGF domains are followed by a cysteine-rich domain in Jagged proteins but not in Delta proteins. However, the consequences of these structural differences remain unclear.

Uyttendaele et al. (1996) cloned cDNAs corresponding to the complete coding region of the mouse Notch4 gene. In situ hybridization revealed that Notch4 transcripts are primarily restricted to endothelial cells in embryonic and adult life, suggesting a role for Notch4 during development of vertebrate endothelium.

Li et al. (Genomics. 1998 Jul. 1; 51(1):45-58) reported that the human NOTCH4 gene contains 30 exons and spans approximately 30 kb. They isolated cDNAs corresponding to 6.7-kb NOTCH4(S) and 9.3-kb NOTCH4(L) mRNA isoforms. The predicted protein encoded by NOTCH4(S) is 2,003 amino acids long and contains the characteristic Notch motifs: a signal peptide, 29 epidermal growth factor (EGF)-like repeats, 3 Notch/lin-12 repeats, a transmembrane region, 6 cdc 10 (603151)/ankyrin repeats, and the PEST conserved region at the C terminus. The sequences of the mouse and human NOTCH4 proteins are 82% identical. The incompletely spliced NOTCH4(L) cDNA potentially encodes 2 different proteins. One consists of the first 7 EGF repeats. The second contains the transmembrane domain and intracellular region and is similar to the mouse int3 protooncoprotein. Northern blot analysis revealed that NOTCH4(S) is the major transcript and is expressed in a wide variety of tissues.

Krebs et al. (2000) generated Notch4-deficient mice by gene targeting. Embryos homozygous for this mutation developed normally, and homozygous mutant adults were viable and fertile. However, the Notch4 mutation displayed genetic interactions with a targeted mutation of the related Notch1 gene. Both Notch1 mutant and Notch1/Notch4 double mutant embryos displayed severe defects in angiogenic vascular remodeling. Analysis of the expression patterns of genes encoding ligands for Notch family receptors indicated that only the Dll4 gene is expressed in a pattern consistent with that expected for a gene encoding a ligand for the Notch1 and Notch4 receptors in the early embryonic vasculature. Therefore, there is an essential role for the Notch signaling pathway in regulating vascular morphogenesis and remodeling, and indicate that whereas the Notch4 gene is not essential during embryonic development, the Notch4 and Notch1 genes have partially overlapping roles during embryogenesis in mice.

As noted above, the disclosure provides methods for using and identifying agonists and antagonists of Dll4 signaling. Candidate agonists and antagonists will generally be any antibody that binds to, or soluble portions of, proteins involved in the Dll4 signaling pathway, including, for example, Dll4, Notch1, Notch4 and presenilin. Candidate agonists and antagonists may also be small molecules or other agents that bind to or effect members of the pathway. Antisense or RNAi nucleic acids may be used as antagonists of Dll4, Notch1, Notch4 or presenilin or other members of the signaling pathway.

Examples of agents include:

(a) an antibody that binds selectively to Dll4;

(b) an antibody that binds selectively to Notch1;

(c) an antibody that binds selectively to Notch4;

(d) an antibody that binds to Notch1 and Notch4;

(e) a polypeptide monomer comprising a Notch-receptor binding portion of Dll4;

(f) a polypeptide multimer comprising two or more polypeptides comprising a Notch-receptor binding portion of Dll4;

(g) a polypeptide monomer comprising a Dll4-binding portion of Notch1 or Notch4;

(h) a polypeptide multimer comprising two or more polypeptides comprising a Dll4-binding portion of Notch1 or Notch4.

Agents that interfere with presenilin activity or other metalloproteinases (e.g., kuzbanian) are expected to modulate Dll4 signaling. Each of these agents may be assessed for agonist or antagonist activity as described herein.

In certain aspects, the agent is a soluble polypeptide comprising an extracellular domain of a Dll4 protein, e.g., as shown in amino acids 27-531 of SEQ ID NO:1. In a specific embodiment, the Dll4 soluble polypeptide comprises a DSL domain of a Dll4 protein.

As used herein, the subject soluble polypeptides include fragments, functional variants, and modified forms of Dll4 soluble polypeptide. These fragments, functional variants, and modified forms of the subject soluble polypeptides may be tested for activity as agonists or antagonists of Dll4 by assessing effects on arterial or venous phenotype in endothelial cells.

In certain embodiments, isolated fragments of the subject soluble polypeptides can be obtained by screening polypeptides recombinantly produced from the corresponding fragment of the nucleic acid encoding an Dll4. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can modulate Dll4 signaling.

In certain embodiments, a functional variant of an Dll4 soluble polypeptide comprises an amino acid sequence that is at least 90%, 95%, 97%, 99% or 100% identical to residues 27-531 of the amino acid sequence of SEQ ID NO: 1.

In certain embodiments, the present invention contemplates making functional variants by modifying the structure of the subject soluble polypeptide for such purposes as enhancing therapeutic or prophylactic efficacy, or stability (e.g., ex vivo shelf life and resistance to proteolytic degradation in vivo). Modified soluble polypeptides can be produced, for instance, by amino acid substitution, deletion, or addition. For instance, it is reasonable to expect, for example, that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains.

This invention further contemplates a method of generating sets of combinatorial mutants of the Dll4 polypeptides, as well as truncation mutants, and is especially useful for identifying functional variant sequences. The purpose of screening such combinatorial libraries may be to generate, for example, soluble polypeptide variants which can act as agonists or antagonists of Dll4. Combinatorially-derived variants can be generated which have a selective potency relative to a naturally occurring soluble polypeptide. Such variant proteins, when expressed from recombinant DNA constructs, can be used in gene therapy protocols. Likewise, mutagenesis can give rise to variants which have intracellular half-lives dramatically different than the corresponding wild-type soluble polypeptide. For example, the altered protein can be rendered either more stable or less stable to proteolytic degradation or other cellular process which result in destruction of, or otherwise inactivation of the protein of interest (e.g., a soluble polypeptide). Such variants, and the genes which encode them, can be utilized to alter the subject soluble polypeptide levels by modulating their half-life. A short half-life can give rise to more transient biological effects and, when part of an inducible expression system, can allow tighter control of recombinant soluble polypeptide levels within the cell. As above, such proteins, and particularly their recombinant nucleic acid constructs, can be used in gene therapy protocols.

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential soluble polypeptide sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, soluble polypeptide variants (e.g., the antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of the subject soluble polypeptide.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of the subject soluble polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, the soluble polypeptides of the invention may further comprise post-translational modifications. Such modifications include, but are not limited to, acetylation, carboxylation, glycosylation, phosphorylation, lipidation, and acylation. As a result, the modified soluble polypeptides may contain non-amino acid elements, such as polyethylene glycols, lipids, poly- or mono-saccharide, and phosphates. Effects of such non-amino acid elements on the functionality of a soluble polypeptide may be tested for its agonist or antagonist effects on Dll4.

In certain aspects, functional variants or modified forms of the subject soluble polypeptides include fusion proteins having at least a portion of the soluble polypeptide and one or more fusion domains. Well known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt- conjugated resins are used. Another fusion domain well known in the art is green fluorescent protein (GFP). Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, the soluble polypeptides of the present invention contain one or more modifications that are capable of stabilizing the soluble polypeptides. For example, such modifications enhance the in vitro half life of the soluble polypeptides, enhance circulatory half life of the soluble polypeptides or reducing proteolytic degradation of the soluble polypeptides.

In certain embodiments, soluble polypeptides (unmodified or modified) of the invention can be produced by a variety of art-known techniques. For example, such soluble polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the soluble polypeptides, fragments or variants thereof may be recombinantly produced using various expression systems as is well known in the art (also see below).

In certain aspects, the invention relates to isolated and/or recombinant nucleic acids encoding a Dll4 polypeptide. The subject nucleic acids may be single-stranded or double-stranded, DNA or RNA molecules. These nucleic acids are useful as therapeutic agents. For example, these nucleic acids are useful in making recombinant soluble polypeptides which are administered to a cell or an individual as therapeutics. Alternative, these nucleic acids can be directly administered to a cell or an individual as therapeutics such as in gene therapy.

In certain embodiments, the invention provides isolated or recombinant nucleic acid sequences that are at least 80%, 85%, 90%, 95%, 97%, 98%, 99% or 100% identical to a region of the nucleotide sequence depicted in SEQ ID NO:2. One of ordinary skill in the art will appreciate that nucleic acid sequences complementary to the subject nucleic acids, and variants of the subject nucleic acids are also within the scope of this invention. In further embodiments, the nucleic acid sequences of the invention can be isolated, recombinant, and/or fused with a heterologous nucleotide sequence, or in a DNA library.

In other embodiments, nucleic acids of the invention also include nucleotide sequences that hybridize under highly stringent conditions to the nucleotide sequence depicted in SEQ ID NO:2, or complement sequences thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from the subject nucleic acids due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

In certain embodiments, the recombinant nucleic acids of the invention may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspect of the invention, the subject nucleic acid is provided in an expression vector comprising a nucleotide sequence encoding a Dll4 polypeptide and operably linked to at least one regulatory sequence. Regulatory sequences are art-recognized and are selected to direct expression of the soluble polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a soluble polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast at-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

This invention also pertains to a host cell transfected with a recombinant gene including a coding sequence for one or more of the subject soluble polypeptide. The host cell may be any prokaryotic or eukaryotic cell. For example, a soluble polypeptide of the invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject soluble polypeptides. For example, a host cell transfected with an expression vector encoding a Dll4 soluble polypeptide can be cultured under appropriate conditions to allow expression of the Dll4 soluble polypeptide to occur. The Dll4 soluble polypeptide may be secreted and isolated from a mixture of cells and medium containing the soluble polypeptides. Alternatively, the soluble polypeptides may be retained cytoplasmically or in a membrane fraction and the cells harvested, lysed and the protein isolated. A cell culture includes host cells, media and other byproducts. Suitable media for cell culture are well known in the art. The soluble polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the soluble polypeptides. In a preferred embodiment, the soluble polypeptide is a fusion protein containing a domain which facilitates its purification.

A recombinant nucleic acid of the invention can be produced by ligating the cloned gene, or a portion thereof, into a vector suitable for expression in either prokaryotic cells, eukaryotic cells (yeast, avian, insect or mammalian), or both. Expression vehicles for production of a recombinant soluble polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant SLC5A8 polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

In certain aspects, the the present invention provides antibodies that have agonist or antagonist effects on Dll4 signaling. Such antibodies may bind to antigens such as Dll4, Notch1 or Notch4. Preferably, the antibody binds to an extracellular domain of such antigens. It is understood that antibodies may be polyclonal or monoclonal; intact or truncated, e.g., F(ab′)2, Fab, Fv; xenogeneic, allogeneic, syngeneic, fully human or modified forms thereof, e.g., humanized, chimeric. Fully human antibodies may be selected from transgenic animals that express human immunoglobulin genes or assembled from recombinant libraries expressing antibody fragments.

For example, by using immunogens derived from Dll4, Notch1 or Notch4, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of the peptide. (e.g., a polypeptide or an antigenic fragment which is capable of eliciting an antibody response, or a fusion protein). Techniques for conferring immunogenicity on a protein or peptide include conjugation to carriers or other techniques well known in the art. An immunogenic portion of an antigen can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with Dll4, Notch1, Notch4 or other target polypeptide and monoclonal antibodies isolated from a culture comprising such hybridoma cells.

The term antibody as used herein is intended to include fragments thereof which are also specifically reactive with antigen. Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. The antibody of the present invention is further intended to include bispecific, single-chain, and chimeric and humanized molecules having affinity for antigen conferred by at least one CDR region of the antibody. Techniques for the production of single chain antibodies (U.S. Pat. No. 4,946,778) can also be adapted to produce single chain antibodies. Also, transgenic mice or other organisms including other mammals, may be used to express humanized antibodies. In preferred embodiments, the antibodies further comprise a label attached thereto and able to be detected (e.g., the label can be a radioisotope, fluorescent compound, enzyme or enzyme co-factor).

In certain preferred embodiments, an antibody of the invention is a monoclonal antibody, and in certain embodiments the invention makes available methods for generating novel antibodies. For example, a method for generating a monoclonal antibody that binds specifically to Dll4, Notch1 or Notch4 may comprise administering to a mouse an amount of an immunogenic composition comprising the antigen polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the antigen. Once obtained, a hybridoma can be propagated in a cell culture, optionally in culture conditions where the hybridoma-derived cells produce the monoclonal antibody. The monoclonal antibody may be purified from the cell culture.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g., by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g. the Biacore binding assay, Bia-core AB, Uppsala, Sweden), sandwich assays (e.g. the paramagnetic bead system of IGEN International, Inc., Gaithersburg, Md.), western blots, immunoprecipitation assays and immunohistochemistry.

In certain aspects, the disclosure provides isolated nucleic acid compounds comprising at least a portion that hybridizes to a Dll4 transcript under physiological conditions and decreases the expression of Dll4 in a cell. Such nucleic acids may be used as Dll4 antagonists, as described herein. The Dll4 transcript may be any pre-splicing transcript (i.e., including introns), post-splicing transcript, as well as any splice variant. In certain embodiments, the Dll4 transcript has a sequence corresponding to the cDNA set forth in SEQ ID NO:2, and particularly the coding portion thereof. In certain aspects, the disclosure provides isolated nucleic acid compounds comprising at least a portion that hybridizes to a Notch1 or Notch4 transcript under physiological conditions and decreases the expression of Notch1 or Notch4 in a cell. These may be used as Dll4 antagonists also. The Notch1 or Notch4 transcript may be any pre-splicing transcript (i.e., including introns), post-splicing transcript, as well as any splice variant.

Examples of categories of nucleic acid compounds include antisense nucleic acids, RNAi constructs and catalytic nucleic acid constructs. A nucleic acid compound may be single or double stranded. A double stranded compound may also include regions of overhang or non-complementarity, where one or the other of the strands is single stranded. A single stranded compound may include regions of self-complementarity, meaning that the compound forms a so-called “hairpin” or “stem-loop” structure, with a region of double helical structure. A nucleic acid compound may comprise a nucleotide sequence that is complementary to a region consisting of no more than 1000, no more than 500, no more than 250, no more than 100 or no more than 50 nucleotides of the Dll4, Notch1 or Notch4 nucleic acid sequence. The region of complementarity will preferably be at least 8 nucleotides, and optionally at least 10 or at least 15 nucleotides. A region of complementarity may fall within an intron, a coding sequence or a noncoding sequence of the target transcript, such as the coding sequence portion. Generally, a nucleic acid compound will have a length of about 8 to about 500 nucleotides or base pairs in length, and optionally the length will be about 14 to about 50 nucleotides. A nucleic acid may be a DNA (particularly for use as an antisense), RNA or RNA:DNA hybrid. Any one strand may include a mixture of DNA and RNA, as well as modified forms that cannot readily be classified as either DNA or RNA. Likewise, a double stranded compound may be DNA:DNA, DNA:RNA or RNA:RNA, and any one strand may also include a mixture of DNA and RNA, as well as modified forms that cannot readily be classified as either DNA or RNA. A nucleic acid compound may include any of a variety of modifications, including one or modifications to the backbone (the sugar-phosphate portion in a natural nucleic acid, including intemucleotide linkages) or the base portion (the purine or pyrimidine portion of a natural nucleic acid). An antisense nucleic acid compound will preferably have a length of about 15 to about 30 nucleotides and will often contain one or more modifications to improve characteristics such as stability in the serum, in a cell or in a place where the compound is likely to be delivered, such as the stomach in the case of orally delivered compounds and the lung for inhaled compounds. In the case of an RNAi construct, the strand complementary to the target transcript will generally be RNA or modifications thereof. The other strand may be RNA, DNA or any other variation. The duplex portion of double stranded or single stranded “hairpin” RNAi construct will preferably have a length of 18 to 40 nucleotides in length and optionally about 21 to 23 nucleotides in length, so long as it serves as a Dicer substrate. Catalytic or enzymatic nucleic acids may be ribozymes or DNA enzymes and may also contain modified forms. Nucleic acid compounds may inhibit expression of the target by about 50%, 75%, 90% or more when contacted with cells under physiological conditions and at a concentration where a nonsense or sense control has little or no effect. Preferred concentrations for testing the effect of nucleic acid compounds are 1, 5 and 10 micromolar. Nucleic acid compounds may also be tested for effects on cellular phenotypes, such as arterial or venous identity.

There are numerous approaches to screening for candidate agents that act as agonists or antagonists of Dll4 signaling. The disclosure provides characteristics that may be used to distinguish agonists and antagonists of Dll4 signaling. In general, agonists of Dll4 signaling stimulate, in a mammalian endothelial cell, expression of an arterial phenotype and inhibit expression of a venous phenotype. In general, antagonists of Dll4 signaling inhibit, in a mammalian endothelial cell, expression of an arterial phenotype and stimulate expression of a venous phenotype. Any known feature that distinguishes arterial and venous endothelial cells may be detected for the purpose of assessing arterial and venous phenotypes. For example, expression of EphrinB2 and expression of connexin37 may be used as indicators of arterial phenotype. As another example, expression of EphB4 may be used as an indicator of venous phenotype.

Agents may also be screened for binding activity to Dll4, Notch1 or Notch4, or for the ability to stimulate or inhibit the production of the active intracellular domain of Notch1 (NICD), Notch4 or Dll4. Expression from hairy/enhancer of split (HES) sensitive promoters may also be useful in determining whether Notch signaling is activated. NICD stimulates expression of HES and HES-driven promoters.

Compounds identified through any screening system can then be tested in animals to assess their effects on angiogenesis, arteriogenesis, or anti-tumor activity in vivo, as well as effects on arterial or venous identity in vivo

High-throughput screening of compounds or molecules can be carried out to identify agents or drugs which inhibit angiogenesis or inhibit tumor growth. Test agents can be any chemical (element, molecule, compound, drug), made synthetically, made by recombinant techniques or isolated from a natural source. For example, test agents can be peptides, polypeptides, peptoids, sugars, hormones, or nucleic acid molecules. In addition, test agents can be small molecules or molecules of greater complexity made by combinatorial chemistry, for example, and compiled into libraries. These libraries can comprise, for example, alcohols, alkyl halides, amines, amides, esters, aldehydes, ethers and other classes of organic compounds. Test agents can also be natural or genetically engineered products isolated from lysates or growth media of cells—bacterial, animal or plant—or can be the cell lysates or growth media themselves. Presentation of test compounds to the test system can be in either an isolated form or as mixtures of compounds, especially in initial screening steps.

For example, an assay can be carried out to screen for compounds that specifically inhibit binding of Dll4 (ligand) to Notch1/Notch4(receptor), or vice-versa, e.g., by inhibition of binding of labeled ligand- or receptor-Fc fusion proteins to immortalized cells.

In one embodiment of an assay to identify a substance that interferes with interaction of two cell surface molecules (e.g., Notch1 and Dll4), samples of cells expressing one type of cell surface molecule are contacted with either labeled ligand or labeled ligand plus a test compound (or group of test compounds). The amount of labeled ligand which has bound to the cells is determined. A lesser amount of label (where the label can be, for example, a radioactive isotope, a fluorescent or colormetric label) in the sample contacted with the test compound(s) is an indication that the test compound(s) interferes with binding. The reciprocal assay using cells expressing a ligand can be used to test for a substance that interferes with the binding of an Eph receptor or soluble portion thereof.

An assay to identify a substance which interferes with interaction between Dll4 and Notch1/Notch4 can be performed with the component (e.g., cells, purified protein, including fusion proteins and portions having binding activity) which is not to be in competition with a test compound, linked to a solid support. The solid support can be any suitable solid phase or matrix, such as a bead, the wall of a plate or other suitable surface (e.g., a well of a microtiter plate), column pore glass (CPG) or a pin that can be submerged into a solution, such as in a well. Linkage of cells or purified protein to the solid support can be either direct or through one or more linker molecules.

In one embodiment, an isolated or purified protein can be immobilized on a suitable affinity matrix by standard techniques, such as chemical cross-linking, or via an antibody raised against the isolated or purified protein, and bound to a solid support. The matrix can be packed in a column or other suitable container and is contacted with one or more compounds (e.g., a mixture) to be tested under conditions suitable for binding of the compound to the protein. For example, a solution containing compounds can be made to flow through the matrix. The matrix can be washed with a suitable wash buffer to remove unbound compounds and non-specifically bound compounds. Compounds which remain bound can be released by a suitable elution buffer. For example, a change in the ionic strength or pH of the elution buffer can lead to a release of compounds. Alternatively, the elution buffer can comprise a release component or components designed to disrupt binding of compounds (e.g., one or more ligands or receptors, as appropriate, or analogs thereof which can disrupt binding or competitively inhibit binding of test compound to the protein).

Fusion proteins comprising all, or a portion of, a protein linked to a second moiety not occurring in that protein as found in nature can be prepared for use in another embodiment of the method. Suitable fusion proteins for this purpose include those in which the second moiety comprises an affinity ligand (e.g., an enzyme, antigen, epitope). The fusion proteins can be produced by inserting the protein or a portion thereof into a suitable expression vector which encodes an affinity ligand. The expression vector can be introduced into a suitable host cell for expression. Host cells are disrupted and the cell material, containing fusion protein, can be bound to a suitable affinity matrix by contacting the cell material with an affinity matrix under conditions sufficient for binding of the affinity ligand portion of the fusion protein to the affinity matrix.

In one aspect of this embodiment, a fusion protein can be immobilized on a suitable affinity matrix under conditions sufficient to bind the affinity ligand portion of the fusion protein to the matrix, and is contacted with one or more compounds (e.g., a mixture) to be tested, under conditions suitable for binding of compounds to the receptor or ligand protein portion of the bound fusion protein. Next, the affinity matrix with bound fusion protein can be washed with a suitable wash buffer to remove unbound compounds and non-specifically bound compounds without significantly disrupting binding of specifically bound compounds. Compounds which remain bound can be released by contacting the affinity matrix having fusion protein bound thereto with a suitable elution buffer (a compound elution buffer). In this aspect, compound elution buffer can be formulated to permit retention of the fusion protein by the affinity matrix, but can be formulated to interfere with binding of the compound(s) tested to the receptor or ligand protein portion of the fusion protein. For example, a change in the ionic strength or pH of the elution buffer can lead to release of compounds, or the elution buffer can comprise a release component or components designed to disrupt binding of compounds to the receptor or ligand protein portion of the fusion protein (e.g., one or more ligands or receptors or analogs thereof which can disrupt binding of compounds to the receptor or ligand protein portion of the fusion protein). Immobilization can be performed prior to, simultaneous with, or after contacting the fusion protein with compound, as appropriate. Various permutations of the method are possible, depending upon factors such as the compounds tested, the affinity matrix selected, and elution buffer formulation. For example, after the wash step, fusion protein with compound bound thereto can be eluted from the affinity matrix with a suitable elution buffer (a matrix elution buffer). Where the fusion protein comprises a cleavable linker, such as a thrombin cleavage site, cleavage from the affinity ligand can release a portion of the fusion with compound bound thereto. Bound compound can then be released from the fusion protein or its cleavage product by an appropriate method, such as extraction.

In certain embodiments, the present invention provides methods of inhibiting angiogenesis and methods of treating angiogenesis-associated diseases. In other embodiments, the present invention provides methods of inhibiting or reducing tumor growth and methods of treating an individual suffering from cancer. These methods involve administering to the individual a therapeutically effective amount of one or more modulators of Dll4 signaling as described above. These methods are particularly aimed at therapeutic and prophylactic treatments of animals, and more particularly, humans.

As described herein, angiogenesis-associated diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; inflammatory disorders such as immune and non-immune inflammation; chronic articular rheumatism and psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome, corneal diseases, rubeosis, arthritis, diabetic neovascularization.

In particular, therapeutic agents of the present invention are useful for treating or preventing a cancer (tumor), including, but not limited to, colon carcinoma, breast cancer, mesothelioma, prostate cancer, bladder cancer, squamous cell carcinoma of the head and neck (HNSCC), Kaposi sarcoma, and leukemia.

In certain embodiments of such methods, one or more therapeutic agents can be administered, together (simultaneously) or at different times (sequentially). In addition, therapeutic agents can be administered with another type of compounds for treating cancer or for inhibiting angiogenesis.

In certain embodiments, the subject methods of the invention can be used alone. Alternatively, the subject methods may be used in combination with other conventional anti-cancer therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through the use of a subject polypeptide therapeutic agent.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When a therapeutic agent of the present invention is administered in combination with another conventional anti-neoplastic agent, either concomitantly or sequentially, such therapeutic agent is shown to enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant cells.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “molecules,” such as bFGF (basic fibroblast growth factor); (2) neutralizers of angiogenic molecules, such as an anti-βbFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. For additional proposed inhibitors of angiogenesis, see Blood et al., Bioch. Biophys. Acta., 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, and 6573256. In addition, there are a wide variety of compounds that can be used to inhibit angiogenesis, for example, peptides or agents that block the VEGF-mediated angiogenesis pathway, endostatin protein or derivatives, lysine binding fragments of angiostatin, melanin or melanin-promoting compounds, plasminogen fragments (e.g., Kringles 1-3 of plasminogen), tropoin subunits, antagonists of vitronectin αᵥβ₃, peptides derived from Saposin B, antibiotics or analogs (e.g., tetracycline, or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

Depending on the nature of the combinatory therapy, administration of the therapeutic agents of the invention may be continued while the other therapy is being administered and/or thereafter. Administration of the polypeptide therapeutic agents may be made in a single dose, or in multiple doses. In some instances, administration of the therapeutic agents is commenced at least several days prior to the conventional therapy, while in other instances, administration is begun either immediately before or at the time of the administration of the conventional therapy.

In certain embodiments, the disclosure provides methods for stimulating arteriogenesis. Such methods may comprise administering to a subject in need thereof, an effective amount of an agonist of Dll4 signaling. The subject may have or be at risk for an ischemic condition. The subject may have coronary artery disease, including, for example, angina or may have had a myocardial infarction. The subject may have a peripheral artery disease, such as an ischemic event or partial occlusion in a limb, the brain or an organ, such as the kidney. The subject may be diagnosed as being at risk for an ischemic event.

In certain embodiments, the disclosure provides methods for promoting the adoption of arterial characteristics in a blood vessel. Such a method may comprise administering to a blood vessel ex vivo or to a subject in need thereof, an effective amount of an agonist of Dll4 signaling. The blood vessel may be a venous graft, such as a saphenous vein graft, such as may be used in a coronary bypass surgery.

In certain embodiments, the subject therapeutic agents of the present invention are formulated with a pharmaceutically acceptable carrier. Such therapeutic agents can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In certain aspects, the disclosure provides pharmaceutical compostions comprising any of the various nucleic acid compounds targeted to Dll4, Notch1, Notch4 or other members of the pathway. A pharmaceutical composition will generally include a pharmaceutically acceptable carrier.

Pharmaceutical compositions suitable for parenteral administration may comprise one or more polypeptide therapeutic agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more polypeptide therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations of the subject polypeptide therapeutic agents include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of anti-tumor or anti-angiogenesis therapeutic agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject therapeutic agent as an active ingredient.

In solid dosage forms for oral administration (capsules, tablets, pills, dragees, powders, granules, and the like), one or more polypeptide therapeutic agents of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Therapeutic agents of the invention can be administered topically, either to skin or to mucosal membranes such as those on the cervix and vagina. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformnamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The subject polypeptide therapeutic agents may be mixed under sterile conditions with a pharmaceutically acceptable carrier, and with any preservatives, buffers, or propellants which may be required. The ointments, pastes, creams and gels may contain, in addition to a subject polypeptide agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a subject polypeptide therapeutic agent, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Formulations for intravaginal or rectal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

In other embodiments, polypeptide therapeutic agents of the instant invention can be expressed within cells from eukaryotic promoters. For example, a soluble polypeptide of Dll4 or Notch1/Notch4 can be expressed in eukaryotic cells from an appropriate vector. The vectors are preferably DNA plasmids or viral vectors. Viral vectors can be constructed based on, but not limited to, adeno-associated virus, retrovirus, adenovirus, or alphavirus. Preferably, the vectors stably introduced in and persist in target cells. Alternatively, viral vectors can be used that provide for transient expression. Such vectors can be repeatedly administered as necessary. Delivery of vectors encoding the subject polypeptide therapeutic agent can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Dosage-Sensitive Requirement for Mouse Dll4 in Artery Development

Duarte et al. (Genes Dev. 2004 Oct. 15; 18(20):2474-8) demonstrated that loss-of-function mutations in mouse Dll4 cause defects in vasculogenesis and angiogenesis, and that these defects are dosage dependent, with Dll4^{+/-} mice showing a less severe phenotype that homozygous Dll4^{-/-} mice. Additionally, the loss of Dll4 function causes a loss of arterial vessel identity. These results demonstrate a level of sensitivity to Dll4 signaling that is unprecedented in the Notch pathway. The sensitivity of vascular development to Dll4 dosage indicates that antagonists of the Dll4-Notch1/4 signaling pathway will be highly effective in inhibiting angiogenesis.

### Example 2

### mDLL4 Overexpression Causes Arterial Hypertrophy and Loss of Venous Identity in Developing Mouse Embryos

In this study Applicants set out to further investigate the role of mDll4 in mammalian vascular development by producing and characterizing murine gain-of- function mutants. To achieve generalized overexpression of mDll4, conditional transgenic mouse lines, ZEG-mDll4, were produced. When crossed with a constitutive cre line, CAG-Cre mice (Sakai et al., 1997), these mice express the native form of mDll4 under the control of the chick beta actin promoter and CMV enhancer. What follows is the description of the gain-of function phenotype observed.

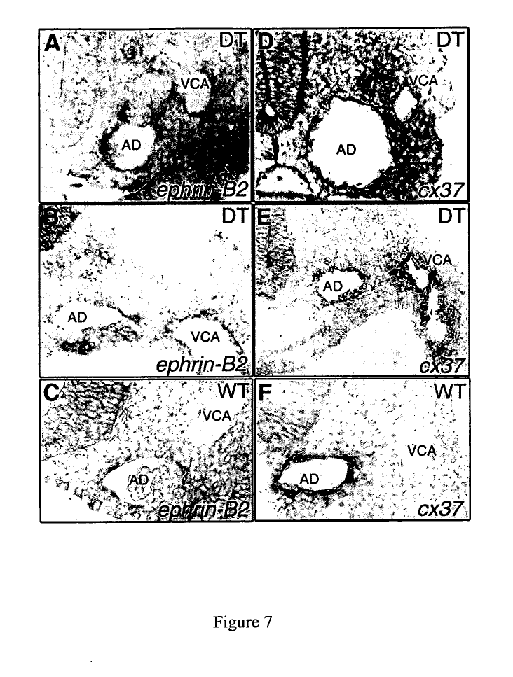

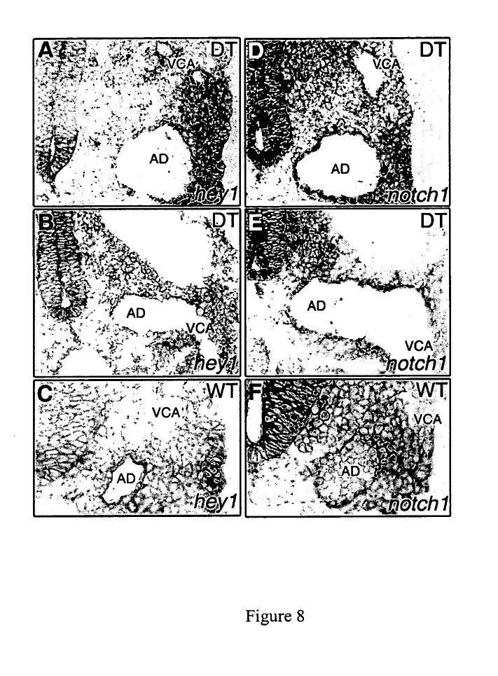

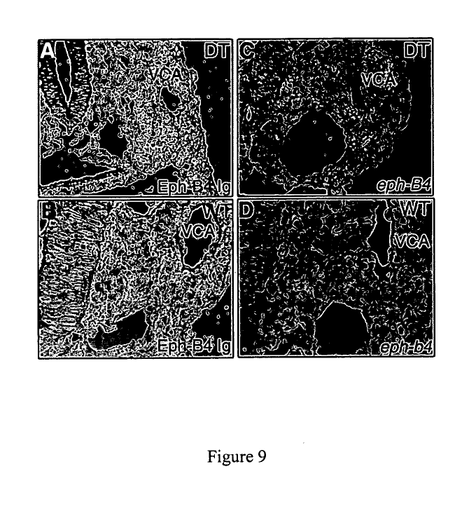

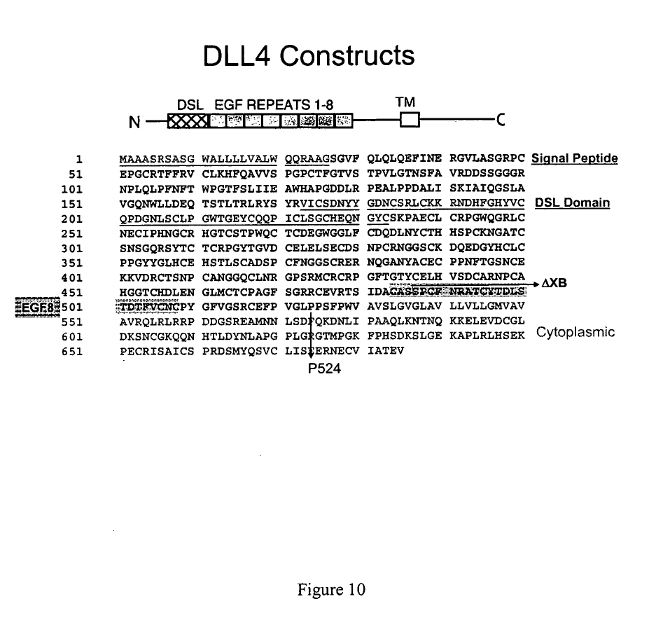

The mDll4 cDNA was cloned in pCALL2-MigR (Lobe et al., 1999) to produce the pZ/EG-mDll4 transgenesis vector (FIG. 7), which was electroporated into R1 mouse embryonic stem (ES) cells. The transgenic mouse lines, derived from electroporated ES cells by standard methods (Nagy & Rossant, 2000), were crossed to the constitutive cre line, CAG-Cre. Resulting embryos were analysed for EGFP fluorescence, the secondary reporter, which is co-expressed with mDll4 in those cells where the Cre recombination has taken place. EGFP expression was found to be strong and generalized in double transgenic embryos (dt) (FIG. 8), which occurred in normal Mendelian ratios at E8.5 through E9.5. These embryos displayed severe haemorrhaging in the head, heart, branchial arches and posterior ventral region, pericardial edema and incomplete turning at E9.0 (FIG. 8c). After E10.5 no double-transgenic embryos were recovered. Immunostaining with PECAM1 antisera in wholemount and cryosections of double transgenic embryos revealed arteriovenous malformations, in particular fusions between the dorsal aortae and the anterior cardinal veins (FIG. 10), as early as E8.5. At this stage there was also an evident degree of aortic hypertrophy, which became progessively more pronounced until E9.5, suggesting a role for the Notch pathway in the regulation of endothelial cell proliferation (FIG. 9). In line with this hypothesis, BrdU incorporation studies showed a 40% average increase in endothelial cell proliferation at the anterior dorsal aortae of the double transgenic embryos (not shown). The anterior cardinal veins (ACV), on the other hand, appeared ramified as if they had not undergone correct angiogenic remodelling (FIG. 9) or nearly absent, except for the region directly connecting to the sinus venosus (FIG. 10). Angiogenic remodelling has also failed to occur in the yolk sac and in the head region, the primary capillary plexus persisting in both cases (FIG. 9). The intersomitic vessels were slightly enlarged and shorter than normal, probably as a consequence of fusions between the arterial and venous vessels and failure to ramify at the dorsal-most extremity (FIG. 9). They were also occasionally seen invading somites, which suggests a disruption of their growth path orientation (not shown). At E9.5, the aortae of dt embryos were seen connecting directly to the sinus venosus region (FIG. 10), creating an arterial microcirculation between the extremities of the heart. Presumably as a consequence of insufficient blood flow, the aorta is severely atrophied posteriorly to its connection to the sinus venosus (FIG. 9 and 10). Microangiography studies with India ink injections show that in these mutants the blood flows from the aortic arches to the aortae and then directly into the sinus venosus, with almost no blood reaching the posterior dorsal aortae (FIG. 10).

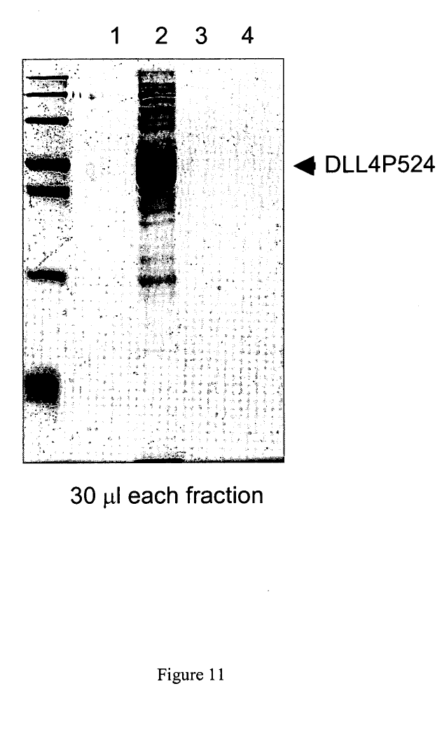

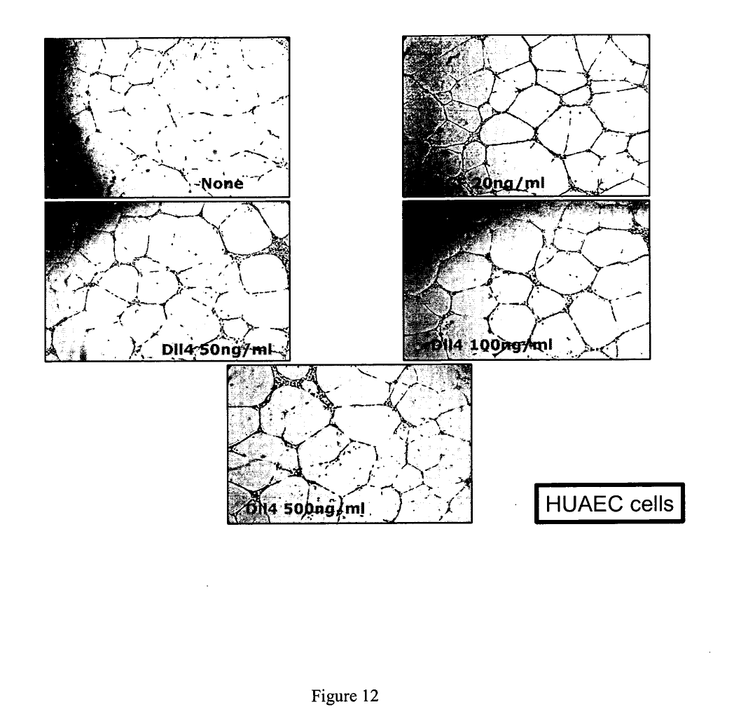

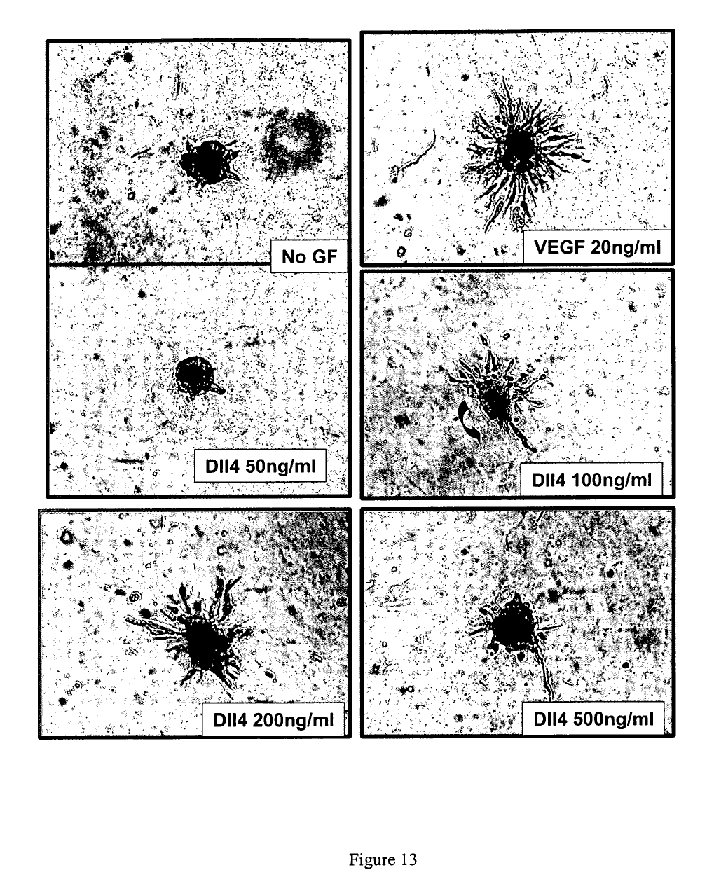

The characterization of arterial- and venous-specific marker expression revealed a striking mutant phenotype, in which all blood vessels in the embryo presented exclusively arterial markers. The veins of the double transgenic embryos showed ectopic expression of arterial-specific markers such as ephrin-B2, connexin37, hey1 and notch1 (FIG. 11 and 12). Expression of venous markers (such as EphB4), on the other hand, could not be detected in these mutants (FIG. 13). Furthermore, hematopoietic clusters, which are normally specific of major arteries in the aorta-gonad-mesonephros region, were detected in the sinus venosus region of the mutant embryos at E9.0 (not shown), providing evidence for functional arteriolization of venous structures.

mDll4 overexpression causes aortic hypertrophy and arteriovenous shunting, localized angiogenic arrest, ectopic expression of endothelial arterial identity markers in venous vessels and downregulation of endothelial venous identity markers.

These results demonstrate a role for mDll4 in the establishment of the endothelial arterial cell identity, and suggest its involvement in the regulation of angioblast/endothelial cell proliferation and angiogenesis.

### Example 3

### Human Delta-Like (hdll4) Regulates Endothelial Cell Tube Formation and Endothelial Cell Sprouting

The extacellular domain of human dll4 (amino acids 26-524 of SEQ ID NO:1) was cloned in mammalian expression vector with His tag or Fc tag, and expressed as secreted protein in 293 cell and CHO-K cells. Purified dll4 was first shown to bind Notch 1 and then tested for its in vitro activity on endothelial cell when cultivated on matrigel. See FIGS. 10 and 11. Endothelial cells organized to make tube like structures within 8-24 hr, and the tube formation was minimal in growth factor deprived condition. Addition of VEGF at 20 ng/ml induces tube formation. Dll4 when tested in growth factor deficient conditions, induced tube formation at lower dose levels (100 and 200 ng/ml) while the higher dose level of 500 ng/ml failed to induce tube formation (FIG. 12). Dll4 however did not induce tube formation when added to VEGF containing cultures.

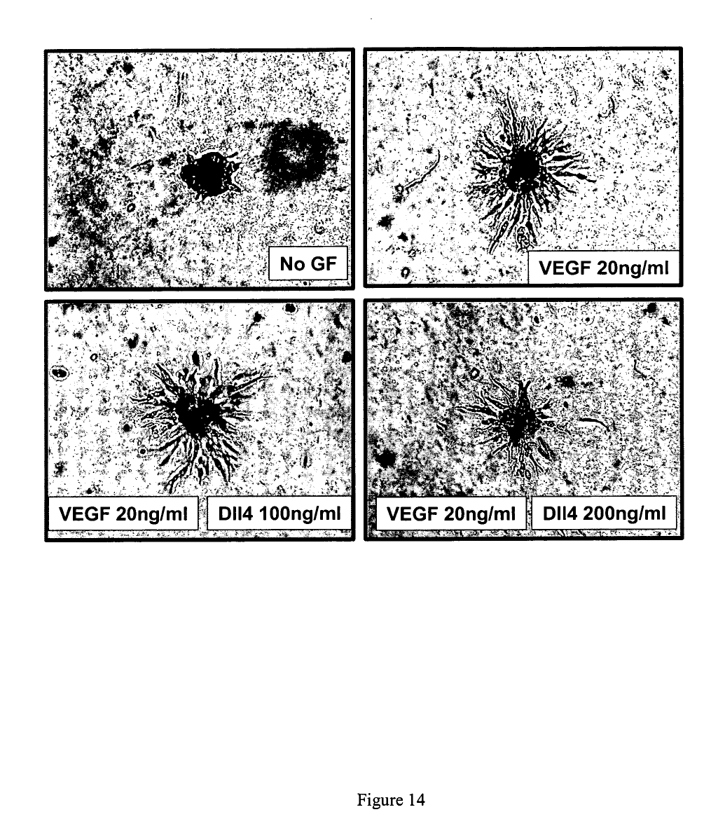

Human dll4 was also tested in sprouting assay where endothelial cell spheroids were treated with varying dose levels of dll4 (FIGS. 13 and 14). Sprouting was induced at the lower dose levels and while higher dose level failed to promote sprouting (FIG. 13). In contrast VEGF treated endothelial cell sprouts were minimally affected by lower levels of dll4, but showed marked reduction in sprouting at higher dose levels (FIG. 14).

### Methods and Materials for Example 3

Generation of Endothelial Cell, Smooth Muscle Cell, and Coculture Spheroids

Endothelial cell and smooth muscle cell spheroids of defined cell number were generated. In brief, SM or HUVE cells were suspended in corresponding culture medium containing 0.25% (w/v) carboxymethylcellulose and seeded in nonadherent round-bottom 96-well plates. Under these conditions, all suspended cells contribute to the formation of a single spheroid per well of defined size and cell number (standard size: 2250 cells/spheroid; in vitro angiogenesis: 750-1000 cells/spheroid). To generate coculture spheroids, equal amounts of suspended SM and HUVE cells (standard size: 1125 SMC and 1125 HUVEC per spheroid; in vitro angiogenesis: 500 SMC and 500 HUVEC per spheroid) were mixed and seeded in nonadherent round-bottom 96-well plates as described above. Spheroids were cultured for at least 24 h and used for the corresponding experiments.

In Vitro Angiogenesis Assay

In vitro angiogenesis in collagen gels was quantitated using endothelial cell, smooth muscle cell, and coculture spheroids as described previously. In brief, spheroids containing 750-1000 cells were generated overnight, after which they were embedded into collagen gels. A collagen stock solution was prepared prior to use by mixing 8 vol acidic collagen extract of rat tails (equilibrated to 2 mg/ml, 4° C.) with 1 vol 10× EBSS (Gibco BRL, Eggenstein, Germany); 1 vol 0.1 N NaOH to adjust the pH to 7.4. This stock solution (0.5 ml) was mixed with 0.5 ml room temperature medium (ECGM basal medium [PromoCell] with 40% FCS containing 0.5% (w/v) carboxymethylcellulose to prevent sedimentation of spheroids prior to polymerization of the collagen gel, 50 spheroids, and the corresponding test substance. The spheroid containing gel was rapidly transferred into prewarmed 24-well plates and allowed to polymerize (1 min), after which 0.1 ml ECGM basal medium was pipetted on top of the gel. The gels were incubated at 37° C., 5% CO2, and 100% humidity. After 24 h, in vitro angiogenesis was digitally quantitated by measuring the length of the sprouts that had grown out of each spheroid (ocular grid at 100× magnification) using the digital imaging software DP-Soft (Olympus) analyzing at least 10 spheroids per experimental group and experiment.

Fluorescent Cell Labeling

SMC and HUVEC were labeled using the fluorescent dyes PKH26 (red fluorescence) and PKH67 (green fluorescence) following manufacturer's instructions. After trypsinization, suspended cells were washed once with HBSS, membrane labeled with PKH26 or PKH67 for 5 min, and washed three times using corresponding culture medium. Quality of cell labeling was examined using fluorescence microscopy.

Ultrastructural Analysis

Spheroids were fixed in Karnovsky's fixative, postfixed in 1.0% osmium tetroxide, dehydrated in a graded series of ethanol, and embedded in Epon. Sections of 0.5 μm were cut and stained with azure 11 methylene blue for light microscopic evaluation. Ultrathin sections (50-80 nm) were cut, collected on copper grids, and automatically stained with uranyl acetate and lead citrate for observation with a Zeiss EM 10 electron microscope.

For quantitation of interendothelial junctional complexes in surface spheroid endothelial cells, all junctional complexes of 20 randomly selected spheroids per experimental group in two independent preparations were counted. Results were expressed as the number of junctional complexes per 100 surface monolayer endothelial cells (analysis of at least 200 EC per experimental group).

Morphological and Immunohistochemical Analysis

Spheroids were harvested and centrifuged for 3 min at 200 g. Cultured monolayer cells were harvested by trypsinization and collected by centrifugation. Spheroids and pelleted monolayer cells were fixed in HBSS containing 4% paraformaldehyde and processed for paraffin embedding; after dehydration (graded series of ethanol and isopropanol, 1 h each), the specimens were first immersed with paraffin I (melting temperature 42° C.) for 12 h at 60° C. Spheroids and monolayer cells were again collected by centrifugation and immersed with paraffin II (melting temperature 56° C.) for 12 h at 70° C. Finally, the resulting paraffin block was cooled to room temperature and trimmed for sectioning. For histochemical analyses, paraffin sections (4 μm) were cut, deparaffinized, and rehydrated. Sections were then incubated with 3% H2O2 in H2O to inhibit endogenous peroxidase. After washings in phosphate-buffered saline, the sections were incubated for 30 min with blocking solution (10% normal goat serum), followed by incubation with the corresponding primary antibody in a humid chamber at 4° C. overnight. Then they were incubated with secondary antibody (biotinylated goat anti-rabbit immunoglobulin or biotinylated goat anti-mouse immunoglobulin antibody; Zymed, San Francisco, Calif.), exposed to streptavidin peroxidase, developed with diaminobenzidine as substrate, and weakly counterstained with hematoxylin.

Detection of Apoptotic Cells in Spheroids

Apoptotic cells were visualized by histochemical detection of nucleosomal fragmentation products (TUNEL) applying the In Situ Cell Death Detection Kit, following the manufacturer's instructions. In brief, nucleosomal fragmentation products in sections of paraffin-embedded spheroids were detected after deparaffination and proteinase K digestion by 3′ end labeling with fluorescein-dUTP using terminal deoxynucleotidyl transferase. Labeling was visualized either directly by fluorescence microscopy or indirectly after incubating the sections with peroxidase-labeled anti-fluorescein antibody and developing with diaminobenzidine as substrate.

DNA Fragmentation Enzyme-Linked Immunoassay (ELISA)

Quantitation of fragmented DNA was performed by ELISA (Cell Death Detection ELISA Kit). Fragmented DNA of 10 spheroids was extracted by lysis for 60 min at room temperature with vigorous shaking. The extracts were centrifuged for 10 min at 13,000 g and 300 μl of the supernatant was incubated with peroxidase-labeled anti-DNA antibody and biotinylated anti-histone antibody in streptavidin-coated microtiter plates following the manufacturer's instructions. After washing, binding of mono- and oligonucleosomal DNA was visualized by developing with the peroxidase substrate ABTS (2,2′-azino-di[3-ethylbenzthiazolin-sulfonate]). Plates were analyzed at 405 nm using an automated microtiter plate reader.

Statistical Analysis

All results are expressed as mean ±SD. Differences between experimental groups were analyzed by unpaired Student's t test. P values <0.05 were considered statistically significant

### REFERENCES

Adams, R. H., Wilkinson, G. A., Weiss, C., Diella, F., Gale, N. W., Deutsch, U., Risau, W. and Klein, R. (1999). Genes Dev 13, 295-306.
Artavanis-Tsakonas, S., Rand, M. D. and Lake, R. J. (1999). Science 284, 770-6.
Duarte, A., Hirashima, M., Benedito, R., Trindade, A., Diniz, P., Bekman, E., Costa, L., Henrique, D., Rossant, J. (2004) Genes Dev 18, 2474-8.
Fischer, A., Schumacher, N., Maier, M., Sendtner, M. and Gessler, M. (2004). Genes Dev 18, 901-11.
Gale, N. W., Baluk, P., Pan, L., Kwan, M., Holash, J., DeChiara, T. M., McDonald, D. M. and Yancopoulos, G. D. (2001). Dev Biol 230, 151-60.
Iso, T., Hamamori, Y. and Kedes, L. (2003) Arterioscler Thromb Vasc Biol 23, 543-53.
Krebs, L. T., Xue, Y., Norton, C. R., Shutter, J. R., Maguire, M., Sundberg, J. P., Gallahan, D., Closson, V., Kitajewski, J., Callahan, R. et al. (2000) Genes Dev 14, 1343-52.
Lawson, N. D., Vogel, A. M. and Weinstein, B. M. (2002) Dev Cell 3, 127-36.
Nagy, A. and Rossant, J. (2000). In Gene targeting: a practical approach, (ed. A. L. Joyner), pp. 177-206. New York: Oxford University Press.
Mumm, J. S. and Kopan, R. (2000) Dev Biol 228, 151-65.
Sakai, K. and Miyazaki, J. (1997) Biochem Biophys Res Commun 237, 318-24.
Shutter, J. R., Scully, S., Fan, W., Richards, W. G., Kitajewski, J., Deblandre, G. A., Kintner, C. R. and Stark, K. L. (2000) Genes Dev 14, 1313-8.
Uyttendaele, H., Marazzi, G., Wu, G., Yan, Q., Sassoon, D. and Kitajewski, J. (1996) Development 122, 2251-9.
Yoneya et al. J. Biochem. Vol. 129, pp. 27-34 (2001)
Li et al. Genomics. 1998 Jul. 1; 51(1):45-58.
Lobe et al., Dev Biol. 1999 Apr. 15; 208(2):281-92.

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for stimulating arteriogenesis, the method comprising, administering to a subject in need thereof, an effective amount of a therapeutic polypeptide comprising an extracellular domain of Dll4.

2. The method of claim 1, wherein the therapeutic polypeptide comprises the DSL domain of SEQ ID NO:1.

3. The method of claim 1, wherein the therapeutic polypeptide comprises amino acids 27-524 of SEQ ID NO:1.

4. The method of claim 1, whereint the therapeutic polypeptide consists of amino acids 27-524 of SEQ ID NO:1 covalently joined to a moiety that confers desirable pharmacokinetic properties.

5. The method of claim 4, wherein the moiety is selected from the group consisting of: an Fc domain or a polyoxyalkylene moiety.

6. The method of claim 1, wherein the therapeutic polypeptide is a monomeric polypeptide.

7. The method of claim 1, wherein the therapeutic polypeptide stimulates, in a mammalian endothelial cell, at an effective concentration, expression of an arterial phenotype.

8. The method of claim 2, wherein the arterial phenotype is selected from the group consisting of: expression of EphrinB2 and expression of connexin37.

9. The method of claim 1, wherein the therapeutic polypeptide inhibits, in a mammalian endothelial cell, at an effective concentration, expression of a venous phenotype.

10. The method of claim 9, wherein the venous phenotype is expression of EphB4.

11. The method of claim 1, wherein the subject has an ischemic condition.

12. The method of claim 1, wherein the subject has coronary artery disease.

13. The method of claim 1, wherein the subject has peripheral artery disease.

14. The method of claim 1, wherein the subject is diagnosed as being at risk for an ischemic event.

15. A method for promoting the adoption of arterial characteristics in a blood vessel, the method comprising administering to a subject in need thereof, an effective amount of a therapeutic polypeptide comprising an extracellular domain of Dll4.

16. The method of claim 15, wherein the therapeutic polypeptide comprises the DSL domain of SEQ ID NO:1.

17. The method of claim 15, wherein the therapeutic polypeptide comprises amino acids 27-524 of SEQ ID NO:1.

18. The method of claim 15, whereint the therapeutic polypeptide consists of amino acids 27-524 of SEQ ID NO:1 covalently joined to a moiety that confers desirable pharmacokinetic properties.

19. The method of claim 18, wherein the moiety is selected from the group consisting of: an Fc domain or a polyoxyalkylene moiety.

20. The method of claim 15, wherein the blood vessel is a venous graft.

21. The method of claim 20, wherein the venous graft is a saphenous vein graft.

22. A method for inhibiting angiogenesis, the method comprising, administering to a subject in need thereof, an effective amount of a therapeutic polypeptide comprising an extracellular domain of Dll4.

23. The method of claim 22, wherein the therapeutic polypeptide comprises the DSL domain of SEQ ID NO:1.

24. The method of claim 22, wherein the therapeutic polypeptide comprises amino acids 27-524 of SEQ ID NO:1.

25. The method of claim 22, whereint the therapeutic polypeptide consists of amino acids 27-524 of SEQ ID NO:1 covalently joined to a moiety that confers desirable pharmacokinetic properties.

26. The method of claim 25, wherein the moiety is selected from the group consisting of: an Fc domain or a polyoxyalkylene moiety.

27. The method of claim 22, wherein the therapeutic polypeptide inhibits, in a mammalian endothelial cell, at an effective concentration, VEGF-stimulated angiogenesis.

28. The method of claim 22, wherein the subject has an angiogenesis-associated disease.

29. The method of claim 28, wherein the angiogenesis-associated disease is selected from the group consisting of angiogenesis-dependent cancer, benign tumors, inflammatory disorders, chronic articular rheumatism and psoriasis, ocular angiogenic diseases, Osler-Webber Syndrome, myocardial angiogenesis, plaque neovascularization, telangiectasia, hemophiliac joints, angiofibroma, wound granulation, wound healing, telangiectasia psoriasis scleroderma, pyogenic granuloma, cororany collaterals, ischemic limb angiogenesis, rubeosis, arthritis and diabetic neovascularization.

30. The method of claim 22, further including administering at least one additional anti-angiogenesis agent that inhibits angiogenesis in an additive or synergistic manner with the therapeutic polypeptide.

31. A method for evaluating the effects of a test agent on Dll4 signaling, the method comprising:
(a) contacting a cell of endothelial lineage with the test agent; and
(b) detecting a phenotype associated with arterial or venous phenotype,
wherein a test agent that promotes the adoption of an arterial phenotype or an agent that inhibits the adoption of a venous phenotype is an agonist of Dll4 signaling, and wherein a test agent that inhibits the adoption of an arterial phenotype or promotes the adoption of a venous phenotype is an antagonist of Dll4 signaling.

32. The method of claim 27, wherein the test agent is selected from the group consisting of:
(a) an antibody that binds selectively to Dll4;
(b) an antibody that binds selectively to Notch1;
(c) an antibody that binds selectively to Notch4;
(d) an antibody that binds to Notch1 and Notch4;
(e) a polypeptide monomer comprising a Notch-receptor binding portion of Dll4;
(f) a polypeptide multimer comprising two or more polypeptides comprising a Notch-receptor binding portion of Dll4;
(g) a polypeptide monomer comprising a Dll4-binding portion of Notch1 or Notch4;
(h) a polypeptide multimer comprising two or more polypeptides comprising a Dll4-binding portion of Notch1 or Notch4.

33. The method of claim 27, wherein the arterial phenotype is selected from the group consisting of: expression of EphrinB2 and expression of connexin37.

34. The method of claim 27, wherein the venous phenotype is expression of EphB4.

35. A method for stimulating arteriogenesis, the method comprising, administering to a subject in need thereof, an effective amount of an agonist of Dll4 signaling.

36. A method for promoting the adoption of arterial characteristics in a blood vessel, the method comprising administering to a subject in need thereof, an effective amount of an agonist of Dll4 signaling.

37. A method for inhibiting angiogenesis, the method comprising, administering to a subject in need thereof, an effective amount of an antagonist of Dll4 signaling.

38. A method for disrupting angiogenesis, the method comprising, administering to a subject in need thereof, an effective amount of an agonist of Dll4 signaling.
